(19) United States
(12) Patent Application Publication
(10) Pub. No.: US 2021/0010076 A1
(43) Pub. Date: Jan. 14, 2021

(54) METHODS AND SYSTEMS FOR ABNORMALITY DETECTION IN THE PATTERNS OF NUCLEIC ACIDS

(71) Applicant: Freenome Holdings, Inc., South San Francisco, CA (US)

(72) Inventors: Daniel DELUBAC, South San Francisco, CA (US); Imran S. HAQUE, San Francisco, CA (US); Michael SINGER, Belmont, CA (US)

(21) Appl. No.: 16/937,287

(22) Filed: Jul. 23, 2020

Related U.S. Application Data
(63) Continuation of application No. PCT/US2019/014740, filed on Jan. 23, 2019.
(60) Provisional application No. 62/621,390, filed on Jan. 24, 2018.

(51) Int. Cl. C12Q 1/6869; G16B 25/00; G16B 40/00

(52) U.S. Cl. CPC C12Q 1/6869; C12Q 2600/156; G16B 40/00; G16B 25/00

(57) ABSTRACT

Systems, media, methods, and kits disclosed herein can improve analysis capabilities of genomic materials. Results from such analyses can be used to detect genomic biomarkers in one or more genomic materials. The systems, media, methods and kits disclosed herein can identify changes or patterns among samples, and can employ machine learning methods to explore changes or potential changes in biological conditions or risks thereof. Further, the systems, media, methods and kits disclosed herein can utilize machine learning algorithms to analyze samples with high accuracy.

### CROSS REFERENCE

This application is a continuation of PCT/US2019/14740, filed Jan. 23, 2019, which claims the benefit of United States Provisional Application No. 62/621,390, filed Jan. 24, 2018, the contents of which are incorporated herein by reference in their entireties.

### BACKGROUND

Genomic biomarkers can be useful for drug discovery and development, and the identification of disease conditions. However, methods of sequencing whole genomes to analyze genomic biomarkers can be time-consuming and prohibitively expensive. Methods of extracting information from genetic material without whole genome sequencing can aid early disease diagnosis, prediction, treatment, and risk stratification.

### SUMMARY

Disclosed herein, in some aspects, are methods for processing a genetic material, such as a nucleic acid sample of a human subject. Processing genetic material can comprise: (a) using a probe set comprising probes having sequencing complementarity with a plurality of regulatory elements to enrich the nucleic acid sample for nucleic acid sequences in the nucleic acid sample comprising at least a subset of the regulatory elements, thereby providing an enriched nucleic acid sample; (b) directing the enriched nucleic acid sample or a derivative thereof to nucleic acid sequencing to generate a plurality of sequence reads comprising sequences that align with sequences from at least a subset of the regulatory elements; (c) computer processing the sequence reads to determine an expression profile of genes corresponding to at least the subset of the regulatory elements; (d) storing the expression profile in a computer memory; optionally (e) analyzing the expression profile using a computer-implemented method; optionally (f) relating a plurality of results of the analysis to a state or condition; and optionally (g) archiving or disseminating the results.

In some aspects, the regulatory elements are deoxyribonucleic acid (DNA) regulatory elements. In some aspects, the DNA regulatory elements are transcriptional start sites (TSS), enhancer sites, silencers, promoters, operators, untranslated regions (UTR), leader sequences (5′ UTR), trailer sequences (3′ UTR), terminators, or any combination thereof. In some aspects, the nucleic acid sample comprises deoxyribonucleic acid (DNA) molecules. In some aspects, the DNA is cell-free DNA. In some aspects, the method further comprises, prior to (b), processing the DNA molecules with a plurality of barcodes. In some aspects, the plurality of barcodes comprise unique molecular identifiers. In some aspects, the regulatory elements are ribonucleic acid (RNA) regulatory elements. In some aspects, the RNA regulatory elements are microRNA (miRNA) regulatory elements, messenger RNA (mRNA) regulatory elements, small interfering RNA (siRNA) regulatory elements, piwi-interacting RNA (piRNA) regulatory elements, small nucleolar RNA (snoRNA) regulatory elements, small nuclear RNA (snRNA) regulatory elements, extracellular RNA (exRNA) regulatory elements, small Cajal body-specific RNA (scaRNA) regulatory elements, non-coding RNA (ncRNA) regulatory elements, or any combination thereof. In some aspects, the nucleic acid sample comprises ribonucleic acid (RNA) molecules. In some aspects, the RNA is cell-free RNA. In some aspects, the method further comprises reverse transcribing the RNA molecules to generate complementary deoxyribonucleic acid molecules. In some aspects, step (c) comprises computer processing the sequence reads against a reference sequence. In some aspects, the reference sequence is from the subject. In some aspects, the reference sequence is from a healthy subject. In some aspects, the reference sequence is an artificial sequence. In some aspects, the reference sequence is derived from a database. In some aspects, step (c) comprises a computer processing method using statistics, mathematics, or biology. In some aspects, the computer processing method is a dimension reduction method. In some aspects, the dimension reduction method is principal component analysis, autoencoding, singular value decomposition, Fourier bases, wavelets, or discriminant analysis.

In some aspects, the computer processing method is a supervised machine learning method. In some aspects, the supervised machine learning method is a regression, support vector machine, tree-based method, neural network, or nearest neighbor method. In some aspects, the computer processing method comprises an unsupervised machine learning method. In some aspects, the unsupervised machine learning method is clustering, neural network, principal component analysis, or matrix factorization. In some aspects, the probe set has an enrichment efficiency for the plurality of regulatory elements that is greater than an enrichment efficiency for other regions of a genome of the subject. In some aspects, the plurality of regulatory elements comprises a first set of regulatory elements having below-average enrichment efficiency and a second set of regulatory elements having above-average enrichment efficiency, and wherein the probe set comprises a first set of probe sequences that targets the first set of regulatory elements and a second set of probe sequences that targets the second set of regulatory elements.

In some aspects, the first set of probe sequences is present at a greater frequency than the second set of probe sequences. In some aspects, the method further comprises analyzing the expression profile using a computer-implemented method. In some aspects, the method further comprises relating results of the analysis to a state or condition. In some aspects, the state or condition is a past, present, or future state or condition. In some aspects, the method further comprises archiving or disseminating the results of the analysis. In some aspects, determining the expression profile comprises determining the availability of the regulatory elements. In some aspects, determining the availability of the regulatory elements comprises quantifying sequencing reads of the regulatory elements. In some aspects, determining the availability of the regulatory elements comprises determining nucleosomal occupancy of the regulatory elements. In some aspects, the method further comprises quantifying a protein level of at least one of the genes. In some aspects, quantifying the protein level comprises performing an immunoassay. In some aspects, nucleic acid sample is from a subject with cancer. In some aspects, nucleic acid sample is from a subject without cancer.

Disclosed herein, in some aspects are systems comprising a computer processor, wherein the computer processor is programmed to: (a) enrich for nucleic acid sequences in a nucleic acid sample from a subject, which nucleic acid sequences comprise at least a subset of regulatory elements, thereby providing an enriched nucleic acid sample; (b) sequence the enriched nucleic acid sample or a derivative thereof to generate a plurality of sequence reads comprising sequences that align with the at least the subset of the regulatory elements; (c) determine an expression profile of genes operably linked to the at least the subset of the regulatory elements; and (d) using at least the expression profile to identify a disease in the subject at an accuracy of at least 90%.

In some aspects, the regulatory elements are deoxyribonucleic acid (DNA) regulatory elements. In some aspects, the DNA regulatory elements are transcriptional start sites (TSS), enhancer sites, silencers, promoters, operators, untranslated regions (UTR), leader sequences (5′ UTR), trailer sequences (3′ UTR), terminators, or any combination thereof. In some aspects, the nucleic acid sample comprises deoxyribonucleic acid (DNA) molecules. In some aspects, the DNA is cell-free DNA. In some aspects, the computer processor is further programmed to, prior to (b), processing the DNA with a plurality of barcodes. In some aspects, the plurality of barcodes comprise unique molecular identifiers. In some aspects, the regulatory elements are ribonucleic acid (RNA) regulatory elements.

In some aspects, the RNA regulatory elements are microRNA (miRNA) regulatory elements, messenger RNA (mRNA) regulatory elements, small interfering RNA (siRNA) regulatory elements, piwi-interacting RNA (piRNA) regulatory elements, small nucleolar RNA (snoRNA) regulatory elements, small nuclear RNA (snRNA) regulatory elements, extracellular RNA (exRNA) regulatory elements, small Cajal body-specific RNA (scaRNA) regulatory elements, non-coding RNA (ncRNA) regulatory elements, or any combination thereof. In some aspects, the nucleic acid sample comprises ribonucleic acid (RNA) molecules. In some aspects, the RNA is cell-free RNA. In some aspects, the computer processor is further programmed to reverse transcribe the RNA molecules to generate complementary deoxyribonucleic acid molecules. In some aspects, step (c) comprises processing the sequence reads against a reference sequence. In some aspects, the reference sequence is from the subject. In some aspects, the reference sequence is from a healthy subject. In some aspects, the reference sequence is an artificial sequence. In some aspects, the reference sequence is derived from a database. In some aspects, the computer processor is further programmed to process the plurality of sequence reads using statistics, mathematics, or biology. In some aspects, processing is a dimension reduction method. In some aspects, the dimension reduction method is principal component analysis, autoencoding, singular value decomposition, Fourier bases, wavelets, or discriminant analysis.

In some aspects, processing is a supervised machine learning method. In some aspects, the supervised machine learning method is a regression, support vector machine, tree-based method, neural network, or nearest neighbor method. In some aspects, processing comprises an unsupervised machine learning method. In some aspects, the unsupervised machine learning method is clustering, neural network, principal component analysis, or matrix factorization. In some aspects, enriching has an enrichment efficiency for the plurality of regulatory elements that is greater than an enrichment efficiency for other regions of a genome of the subject. In some aspects, the plurality of regulatory elements comprises a first set of regulatory elements having below-average enrichment efficiency and a second set of regulatory elements having above-average enrichment efficiency, and wherein the probe set comprises a first set of probe sequences that targets the first set of regulatory elements and a second set of probe sequences that targets the second set of regulatory elements.

In some aspects, the first set of probe sequences are present at a greater frequency than the second set of probe sequences. In some aspects, the computer processor is further programmed to analyze the expression profile using a computer-implemented method. In some aspects, the computer processor is further programmed to relate results of the analysis to a state or condition. In some aspects, the the state or condition is a past, present, or future state or condition. In some aspects, the computer processor is further programmed to archive or disseminate the results of the analysis. In some aspects, the computer processor is further programmed to determine the availability of the regulatory elements.

In some aspects, the computer processor is further programmed to quantify sequencing reads of the regulatory elements. In some aspects, the computer processor is further programmed to determine nucleosomal occupancy of the regulatory elements. In some aspects, the biological sample is from a subject with cancer. In some aspects, the biological sample is from a subject without cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent that publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

### DETAILED DESCRIPTION

While various embodiments of the invention have been shown and described herein, it will be obvious to those having ordinary skill in the art that such embodiments are provided by way of example only. Numerous variations, changes, and substitutions can occur to those having ordinary skill in the art without departing from the invention. It should be understood that various alternatives to the embodiments of the invention described herein can be employed.

### Definitions

As used herein, the term “biological sample” refers to any suitable biological sample that comprises a nucleic acid, a protein, or any other biological analyte. The biological sample may be obtained from a subject. A biological sample may be solid matter (e.g., biological tissue) or a fluid (e.g., a biological fluid). In general, a biological fluid can include any fluid associated with living organisms. Non-limiting examples of a biological sample include blood or components of blood (e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

The term “nucleic acid sample” may encompass “nucleic acid library” or “library” which, as used herein, includes a nucleic acid library that has been prepared by any method known in the art. In some instances, providing the nucleic acid library may include the steps required for preparing the library, for example, including the process of incorporating one or more nucleic acid samples into a vector-based collection, such as by ligation into a vector and transformation of a host. In some instances, providing a nucleic acid library may include the process of incorporating a nucleic acid sample into a non-vector-based collection, such as by ligation to adaptors. The adaptors may anneal to PCR primers to facilitate amplification by PCR or may be universal primer regions such as, for example, sequencing tail adaptors. The adaptors may be universal sequencing adaptors. As used herein, the term “efficiency,” may refer to a measurable metric calculated as the division of the number of unique molecules for which sequences will be available after sequencing over the number of unique molecules originally present in the primary sample. Additionally, the term “efficiency” may also refer to reducing initial nucleic acid sample material required, decreasing sample preparation time, decreasing amplification processes, and/or reducing overall cost of nucleic acid library preparation.

As used herein, the terms “polynucleotide”, “nucleic acid”, and “oligonucleotide” can be used interchangeably. These terms can refer to a polymeric form of nucleotides of any length, either deoxyribonucleotides or ribonucleotides, or analogs thereof. Polynucleotides have any three-dimensional structure. Polynucleotides can perform any function, known or unknown. Non-limiting examples of polynucleotides include coding regions of a gene or gene fragment, non-coding regions of a gene or gene fragment, loci (locus) defined from linkage analysis, exons, introns, messenger RNA (mRNA), transfer RNA, ribosomal RNA, ribozymes, complementary DNA (cDNA), recombinant polynucleotides, branched polynucleotides, plasmids, vectors, isolated DNA of any sequence, isolated RNA of any sequence, nucleic acid probes, and primers. RNA can be reverse transcribed to generate cDNA. A polynucleotide can include modified nucleotides, such as methylated nucleotides and nucleotide analogs. If present, modifications to the nucleotide structure can be imparted before or after assembly of the polymer. A sequence of nucleotides can be interrupted by non-nucleotide components. A polynucleotide can be further modified after polymerization, such as by conjugation with a labeling component.

As used herein, the term “subject,” generally refers to an entity or a medium that has testable or detectable biological information. A biological sample can be obtained from a subject. A subject can be a person or individual. A subject can be an invertebrate or a vertebrate, such as, for example, a mammal. Non-limiting examples of mammals include murines, simians, humans, farm animals, sport animals, and pets.

As used herein, the term “healthy” refers to a biological sample or subject that not suspected or does not have a disease, not known to have a disease, or not known to have previously had a disease. For example, a healthy subject can be a subject that is not suspected or does not have a cancer.

As used herein, the term a “nucleic acid sample” refers to a collection of nucleic acid molecules. In some instances, the nucleic acid sample may be from a single biological source, e.g., one individual or one tissue sample, and in other instances, the nucleic acid sample may be a pooled sample, e.g., containing nucleic acids from more than one organism, individual, or tissue. In some instances, the nucleic acid sample may be a recombinant nucleic acid. Non-limiting examples of synthetic nucleic acids include plasmids, viral vectors, and shRNAs. In some instances, the nucleic acid sample may be a synthetic nucleic acid. Non-limiting examples of synthetic nucleic acids include synthetic RNA such as RNA spike-ins, synthetic DNA such as sequins, primers, and modified analogs of nucleotides, such as morpholinos and siRNA.

As used herein, the term “barcode” or “unique molecular identifier (UMI)” may be a known sequence used to associate a polynucleotide fragment with the input polynucleotide or target polynucleotide from which it is produced. It can be a sequence of synthetic nucleotides or natural nucleotides. A barcode sequence may be contained within adapter sequences such that the barcode sequence is contained in the sequencing reads. Each barcode sequence may include at least 4, at least 5, at least 6, at least 7, at least 8, at least 9, at least 10, at least 11, at least 12, at least 13, at least 14, at least 15, at least 16, or more nucleotides in length. In some cases, barcode sequences may be of sufficient length and may be sufficiently different from one another to allow the identification of samples based on barcode sequences with which they are associated. In some cases, barcode sequences are used to tag and subsequently identify an “original” nucleic acid molecule (i.e. a nucleic acid molecule present in a sample from a subject). In some cases, a barcode sequence, or a combination of barcode sequences, is used in conjunction with endogenous sequence information to identify an original nucleic acid molecule. For example, a barcode sequence (or combination of barcode sequences) can be used with endogenous sequences adjacent to the barcodes (e.g., at the beginning and end of the endogenous sequences) and/or with the length of the endogenous sequence.

As used herein, the term “next-generation sequencer” refers to a sequencer which is capable of next-generation sequencing. A next-generation sequencer can include a number of different sequencers, such as Illumina sequencers.

In some embodiments, nucleic acid molecules used herein can be subjected to a “tagmentation” or “ligation” reaction. “Tagmentation” combines the fragmentation and ligation reactions into a single step of the library preparation process. The tagged polynucleotide fragment is “tagged” with transposon end sequences during tagmentation and may further include additional sequences added during extension during a few cycles of amplification. Alternatively, the biological fragment can directly be “tagged,” for example, with ligation adapters, with or without a preceding “end preparation” reaction.

As used herein, the terms “accuracy,” “specificity,” “sensitivity,” and “precision” generally refers to sequencing or base calling accuracy, specificity, sensitivity, or precision, respectively. Accuracy, specificity, sensitivity, and precision are functions of the number of true positive base calls (TP), true negative base calls (TN), false positive base calls (FP), and false negative base calls (FN). A true positive is a base call for a particular base that correctly identifies the base. A true negative is a base call ruling out a particular base that correctly rules out the base. A false positive is a base call for a particular base that incorrectly identifies the base. A false negative is a base call ruling out a particular base that incorrectly rules out the base. Accuracy is measured as (TP +TN)/(TP+TN+FP+FN). Specificity is measured as (TN)/(TN+FP). Sensitivity is measured as (TP)/(TP+FN). Precision is measured as (TP)/(TP+FP). Positive Predictive Value (PPV) is measured as TP/(TP+FP); Negative Predictive Value (NPV) is measured as TN/(TN+FN).

The present disclosure provides systems and methods for characterizing targeted regions of genomic material for improving cancer diagnostics. In some embodiments, the disclosure relates to systems and methods for analyzing regulatory elements of whole genomes. Regulatory elements of interest can include DNA regulatory elements and/or RNA regulatory elements. DNA regulatory elements can include, for example, transcriptional start sites (TSS), enhancer sites, silencers, promoters, operators, untranslated regions (UTR), leader sequences (5′ UTR), trailer sequences (3′ UTR), terminators, and any combination thereof RNA regulatory elements can include, for example, microRNA (miRNA) regulatory elements, messenger RNA (mRNA) regulatory elements, small interfering RNA (siRNA) regulatory elements, piwi-interacting RNA (piRNA) regulatory elements, small nucleolar RNA (snoRNA) regulatory elements, small nuclear RNA (snRNA) regulatory elements, extracellular RNA (exRNA) regulatory elements, small Cajal body-specific RNA (scaRNA) regulatory elements, non-coding RNA (ncRNA) regulatory elements, and any combination thereof.

DNA transcriptional regulatory elements can include, for example, core promoters, transcriptional start sites, proximal promoters, enhancers, distal enhancers, silencers, insulators, boundary elements, locus control regions, transcription factors, activators, coactivators, and any combination thereof. In some embodiments, the disclosure relates to systems and methods for analyzing transcriptional start site (TSS) panels of a whole genome.

The whole genome and derivatives thereof (e.g., RNA and proteins), collectively referred to as genomic material, can include many biochemical components. Various laboratory techniques can be used to characterize genomic material, including, for example, genomic sequencing, methylation, small molecule arrays (Simoa™), and enzyme-linked immunosorbent assays (ELISA). Accurate characterization of genetic material can be time-consuming and expensive. The present disclosure therefore provides improved methods of characterizing genomic material by reducing the time and cost of extracting information from genomic materials.

Identification of regulatory elements can aid understanding of how gene expression is altered in pathological conditions and which gene expression patterns are associated with pathological conditions. Regulatory elements can exhibit various characteristics that correlate with a diseased state, wellness state, or pathological condition and/or phenotype. These characteristics include, for example, single nucleotide polymorphisms (SNPs), variability of short sequence repeats, DNA modifications, methylation, acetylation, insertions, deletions, copy number variations, cytogenetic rearrangements, translocations, duplications, deletions, inversions, RNA sequence, RNA expression levels, RNA splicing and editing, mRNA levels, and microRNA levels.

Certain regions of genomic material can have characteristics that have an impact on human characteristics or function, have no impact on human characteristics or function, or have an unknown impact on human characteristics or function. An impact on human characteristics can include, for example, overall well-being, physical state, mental state, and disposition. An impact on human function can include, for example, formation of a pathological feature or structural abnormality, evolution of a pathological feature or structural abnormality, and development of a pathological feature or structural abnormality.

The characteristic or functional impact of a structural or pathological feature can occur through a biological network that involves one or more genomic materials. Characteristics of a biological network can be a function of one or more genomic materials that comprise a portion of or an entire biological network. Genetic material that is involved in a biological network can contain one or more characteristics that impact characteristics and/or pathology. Aspects of one or more components of a biological network can be coupled or can interact with one another to impact characteristics or functions of the biological network. The impacted aspects of the biological network can impact characteristics and/or pathology, and the impact can comprise functional and/or temporal considerations. The biological network can be comprised of biological components that occupy a portion of one or more genomic material or regions of the genome.

Methods can be constructed to obtain one or more specific characteristics of genomic material of a biological network comprised of one or more genomic materials. These methods can be referred to as “targeted methods”. Targeted methods can include, for example, laboratory methods, data analysis methods, computational methods, visualization methods, and usage methods. Targeted methods can include, for example, targeted sequencing (based on amplification or hybridization), digital sequencing, high depth/intensity sequencing, analysis of TSS, analysis of enhancers, and characterization of specific genes. Usage methods can limit the application of targeted methods to specific use cases, which can depend, for example, on clinical indication, operating environment, or intended use.

Targeted methods can alleviate constraints that inhibit a broad collection, analysis, and dissemination of characteristics of genomic material. In addition, targeted methods can alleviate the need for specific types of genomic material, which can be expensive, difficult to obtain, process, or handle. For example, targeted sequencing methods can reduce the cost and time of sequencing the entire genome. Targeted data analysis can alleviate computational burdens (e.g., computer memory and CPU time) of analyzing the entire genome. Targeted computational methods and algorithms, which process only a portion of data contained within a large or complex biological network, can reduce the computational burdens of processing the entire network. The application of targeted methods can enable the acquisition of characteristic or functional information from specific types of genomic materials and can combine or process different aspects of different genomic material using different techniques.

Targeted methods can be applied to one or more genomic materials, to one or more genomic materials that comprise a biological network, or to a biological network as a whole. For example, targeted sequencing can be applied to one or more regions of the genome. Targeted sequencing can comprise sequencing specific genes, non-coding regions or other specific regions of interest within the genome. Targeted assays can be used to characterize one or more proteins, or the interaction between genes or proteins. Genes or proteins can be characterized by measuring expression levels or determining an expression profile. In some embodiments, determining an expression profile comprises determining the availability of regulatory elements, for example, by quantifying sequencing reads of the regulatory elements or determining nucleosomal occupancy of the regulatory elements. By determining whether a regulatory element is available, one of skill in the art can know whether a downstream gene that is operably linked to the regulatory element will be able to be expressed. In some embodiments, the methods of the present disclosure also provide quantifying a protein level of at least one of a gene, e.g., a gene operably linked to a regulatory element. Quantifying a protein level can comprise performing an immunoassay.

Targeted methods can identify and obtain characteristics of genomic material that impact characteristics or pathology. Aspects that impact pathology can include, for example, a single genetic mutation or multiple genetic mutations. Targeted methods can also identify relationships between multiple mutations within the genome that impact pathology. Targeted methods can identify networks of genetic mutations, and similarities and differences amongst networks.

In the context of multi-analyte testing, changes in cfDNA patterns can be correlated with regulatory regions to measure translation, transcription, and regulation. For example, cfDNA-based estimates of expression can be integrated with the direct circulating protein concentration. Moreover, cfDNA-based estimation of regulatory function (enhancer expression or expression of regulatory genes) can be integrated with aspects of miRNA regulatory function. In some embodiments, regulatory and other genomic elements present in circulating DNA or regulatory RNAs can be jointly captured and assayed. These genomic elements can be acquired using targeted methods. Regulatory RNAs can be captured after reverse transcription or direct RNA pulldown. Variable widths can be captured across the TSS or regions of the genome.

The present disclosure provides systems and methods for analyzing panels of regulatory elements from whole genomes. For example, TSS and enhancer panels from cell-free DNA (cfDNA) can provide information about genomic data without whole genome sequencing by using inference methods, methods of statistical or mathematical analysis, or methods of statistical or mathematical modeling. The methods of the present disclosure improve on existing methods of whole genome sequencing by reducing sequencing expenditure by enriching for certain regions of the genome (e.g., regulatory elements). For example, sequencing expenditure can be reduced by selecting targeted regions of genomic material. The targeted regions can include regions of genomic material that are correlated with desired characteristics. Desired characteristics can include aspects related to functional or pathological condition or state. Data quality can be improved by increasing sequencing depth and sampling resolution at constant sequencing cost, thereby reducing time and material resources. In some embodiments, data quality can be improved by compensating for known characteristics. For example, known characteristics can include sequence, length, and epigenetic modifications of the genomic material. In some embodiments, data quality can be improved by selectively enriching or depleting particular captured regions of the genomic material. In some embodiments, data quality can be improved by leveraging information from regulated genes, TSSs, promoters, enhancers, and other regulatory elements. Thus, targeted methods can improve process efficiency for high throughput and process scaling. Targeted methods can also enable scientific discovery by facilitating the acquisition of specific data of a desired quantity, quality, and accuracy.

Targeted methods can include the use of hybridization probes. Hybridization probes can enrich genomic material by detecting fragments of genomic material that are complementary to the sequence of the probe. The probe can hybridize to single-stranded nucleic acid fragments (for example, DNA or RNA) whose base sequence allows probe-target base pairing due to complementarity between the probe and the target sequence. Hybridization probes can thereby enable the acquisition of targeted data. The degree of hybridization may be assayed in a quantitative matter using various methods known in the art. The degree of hybridization at a probe position may be related to the intensity of signal provided by the assay, which is therefore related to the amount of complementary nucleic acid sequence present in the sample. Computer-based software can be used to extract, normalize, summarize, and analyze array intensity data from probes across the human genome or transcriptome, including expressed genes, exons, introns, and miRNAs. In some embodiments, the intensity of a given probe in either the benign or malignant samples can be compared against a reference set to determine whether differential expression is occurring in a sample. An increase or decrease in relative intensity at a marker position on an array corresponding to an expressed sequence is indicative of an increase or decrease respective of expression of the corresponding expressed sequence.

A hybridization probe set of the present disclosure may provide an enrichment efficiency for a set of regulatory elements that is greater than an enrichment efficiency for other regions in a genome of a subject. For example, a plurality of regulatory elements can comprise a first set of regulatory elements having below-average enrichment efficiency and a second set of regulatory elements having above-average enrichment efficiency. The probe set can include a first set of probe sequences that targets the first set of regulatory elements and a second set of probe sequences that targets the second set of regulatory elements.

Targeted sequencing can include barcoding methods. Barcoding methods can entail building a barcode library of known species and matching the barcode sequence of an unknown sample of genomic material against the barcode library for identification. First, a genomic material sample can undergo fragmentation by enzymatic methods. Various different restriction enzymes can be used to generate fragments with some fragments differing in length. The restriction enzymes can have a recognition site of at least about 6 nucleotides in length. Fragments of genomic material can have a median length from about 200 nucleotides to about 10,000 nucleotides. The fragments can then be attached to different barcodes by enzymatic methods. For example, fragments can be barcoded by a ligase. Barcoded fragments can be pooled or unpooled prior to sequencing.

Barcoding can involve the use of unique barcodes or unique molecule identifiers from a barcode library. In some embodiments, barcoding can involve the use of non-unique barcodes. Non-unique barcodes methods can use the endogenous sequence of a fragment for unique identification. For example, a nucleic acid molecule with non-unique barcodes can be identified by a combination of barcode sequences plus the beginning and end of the endogenous sequence adjacent to the barcode.

Hybridization probes can be used to enrich TSS sequences in genomic material. TSSs can be highly regulated by chromatin folding and histone positioning. Information obtained from TSS sequences can provide information about gene expression status and pathology. Panels can reveal various direct information, including, for example, patterns of depth, length, location, position, and sequence of nucleic acid fragments, such as cfDNA fragments. Direct information can subsequently be used to determine indirect information, including, for example, inferred gene expression, inferred nucleosome occupancy, and inferred chromatin changes, without measuring RNA levels or protein levels in a sample. Accordingly, regulatory element panels can be used to assess changes to gene expression and regulatory networks associated with diseases, conditions, age, risk, and health status.

Targeted methods can be “static” (or constant) throughout a laboratory process, “prescribed” (or dynamic) while following a set of instructions, or “adaptive” depending on the progress. A targeted method can comprise one or more laboratory processes that can be “static,” “prescribed,” or “adaptive”. The application of such methods can change during the course of a laboratory process.

Data collected from one or more genomic materials can be characterized by one or more accuracies that describe spatial or temporal fidelity of the data. For example, global accuracy can characterize the bulk accuracy of data collected from genomic materials. Local accuracy can characterize the accuracy of a specific region within genomic materials.

The accuracy of characteristics obtained by targeted methods can be: uniform, wherein the accuracy of a characteristic is constant throughout genomic materials; non-uniform, wherein the accuracy of a characteristic is non-constant throughout genomic materials; or variable, wherein the accuracy of one or more characteristics is different for different characteristics. The accuracy of characteristics obtained by targeted methods can be constant or non-constant throughout the execution of the targeted method.

Acquisition and analysis of data collected from one or more genomic materials or from a network of genomic materials can be dynamic. For example, the accuracy and/or frequency of data collection can change in response to changing biological, environmental, or experimental factors. Accuracy and/or frequency of data collection can change in response to one or more prescribed rules. For example, genomic sequencing can be applied with 5× depth for O-blood type and applied with 10× depth for A-blood type.

Data can be analyzed in a dynamic manner and can depend on the method of data collection, e.g., real-time analysis system with feedback. The order in which data are collected can be dynamic and can depend on various factors, including, for example, method of data collection, type of genomic material, availability of laboratory equipment, and environmental factors. The time required to collect data can be dynamic and can depend on various factors, including, e.g., the type of genomic material, the nature of biological processes, laboratory equipment, and environmental factors.

Targeted methods can characterize one or more aspects within a biological network comprised of one or more genomic materials, e.g., rate(s) at which one or more biological processes occur; aspects of the conversion of genomic material, e.g., amount of RNA transcribed to protein, extent to which genes are expressed, amount of mRNA observed; signals associated with genomic activity, materials, and networks, e.g., the strength/frequency of biochemical signals that can flow within one or more genomic materials and the strength/frequency of biochemical signals that can flow within one or more networks of genomic materials; and correlations or independence amongst targeted regions of genomic materials that comprise biological networks or portions of biological networks.

Targeted methods can characterize the functional significance of genomic materials, e.g., correlations between characteristics of regions of genomic materials; correlations between regions of genomic materials and pathological states; and correlations between characteristics of a network. Targeted methods can be used to identify one or more activation thresholds that characterize the functional significance of one or more regions of the genome or one or more aspects of a biological network. Targeted methods can be used to identify nodes or pathways of a regulatory network, which can comprise regions of one or more genomic materials that lead to pathological states. Targeted methods can be used to identify the mechanisms by which one or more genomic materials impact other genomic materials within a network. Targeted methods can enable diagnosis of medical conditions and the formulation of causal pathways.

The present disclosure provides a method of diagnosing a cancer by determining an expression profile of one or more regulatory elements in the biological sample and identifying the biological sample as cancerous based on the expression profile of the one or more regulatory elements in the biological sample. In some embodiments, the method further includes comparing the expression profile of the one or more regulatory elements to a control expression profile of the one or more regulatory elements in a control sample (i.e. a non-cancerous sample). The biological sample may be identified as cancerous based on a difference in the expression profile between the one or more regulatory elements in the biological sample and the control sample.

In one aspect, the present disclosure provides a method for sequencing a nucleic acid sample to generate one or more sequences of the nucleic acid sample at an efficiency, accuracy, sensitivity, precision, specificity, positive predictive value, or negative predictive value that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

The present disclosure provides a method of diagnosing a cancer with a specificity and/or sensitivity that is at least 70% using methods described herein by comparing the expression profile of one of more regulatory elements in the biological sample with a control sample and identifying the biological sample as cancerous if there is a difference in the expression profile between the biological sample and the control sample at a specified confidence level. In some embodiments, the specificity and/or sensitivity can be at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In some embodiments, the specificity is at least 70%. In some embodiments, the nominal negative predictive value (NPV) is at least 95%. In some embodiments, the NPV is at least 95%, at least 95.5%, at least 96%, at least 96.5%, at least 97%, at least 97.5%, at least 98%, at least 98.5%, at least 99%, at least 99.5%, or more.

Sensitivity can refer to TP/(TP+FN), where TP is true positive and FN is false negative. Specificity typically refers to TN/(TN+FP), where TN is true negative and FP is false positive. The number of benign results divided by the total number of benign results based on adjudicated histopathology diagnosis.

In some embodiments, the difference in gene expression level is at least 10%, at least 15%, at least 20%, at least 25%, at least 30%, at least 35%, at least 40%, at least 45%, at least 50%, or more. In some embodiments, the difference in gene expression level is at least 2-fold, at least 3-fold, at least 4-fold, at least 5-fold, at least 6-fold, at least 7-fold, at least 8-fold, at least 9-fold, at least 10-fold, or more. In some embodiments, the biological sample is identified as cancerous with an accuracy of at least 75%, at least 80%, at least 85%, at least 90%, at least 95%, at least 99%, or more. In some embodiments, the biological sample is identified as cancerous with a sensitivity of at least 95%. In some embodiments, the biological sample is identified as cancerous with a specificity of at least 95%. In some embodiments, the biological sample is identified as cancerous with a sensitivity of at least 95% and a specificity of at least 95%. In some embodiments, the accuracy is calculated using a trained algorithm.

In some embodiments, the gene expression product is a protein, and the amount of protein is compared. The amount of protein can be determined by ELISA, mass spectrometry, blotting, immunohistochemistry, or any combination thereof. RNA can be measured by microarray, serial analysis of gene expression (SAGE), blotting, RT-PCR, quantitative PCR, sequencing (e.g., by RNA-seq), or any combination thereof.

In some embodiments, the difference in gene expression level between a biological sample and a control sample that can be used to diagnose a cancer is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5, at least 9-fold, at least 9.5-fold, at least 10-fold, or more.

In some embodiments, the biological sample is classified as cancerous or positive for a subtype of cancer with an accuracy of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5%. The diagnosis accuracy can include specificity, sensitivity, positive predictive value, negative predictive value, and/or false discovery rate.

When classifying a biological sample for diagnosis of a cancer, there are typically four possible outcomes from a binary classifier. If the outcome from a prediction is p and the actual value is also p, then it is called a true positive (TP). However, if the actual value is n, then it is a false positive (FP). Conversely, a true negative has occurred when both the prediction outcome and the actual value are n, and false negative is when the prediction outcome is n while the actual value is p. As an example, consider a diagnostic test to determine whether a subject has a disease. A false positive occurs when the subject tests positive, but does not actually have the disease. A false negative, on the other hand, occurs when the subject tests negative, suggesting that the subject is healthy, when the subject actually does have the disease. In some embodiments, a receiver operating characteristic (ROC) curve assuming real-world prevalence of subtypes can be generated by re-sampling such errors generated from available samples in relevant proportions.

The positive predictive value (PPV), or precision rate, or post-test probability of disease, is the proportion of subjects with positive test results who are correctly diagnosed. The PPV is an important measure of a diagnostic method as it reflects the probability that a positive test reflects the underlying condition being tested. However, the PPV value depends on the prevalence of the disease, which may vary based on the analysis. For example, FP (false positive); TN (true negative); TP (true positive); FN (false negative).
False positive rate(α)=FP/(FP+TN)−specificity
False negative rate(β)=FN/(TP+FN)−sensitivity
Power=sensitivity=1−β
Likelihood-ratio positive=sensitivity/(1−specificity)
Likelihood-ratio negative=(1−sensitivity)/specificity

The negative predictive value (NPV) is the proportion of subjects with negative test results who are correctly diagnosed. PPV and NPV measurements can be derived using appropriate disease subtype prevalence estimates. An estimate of the pooled disease prevalence can be calculated from the pool of indeterminants. For subtype specific estimates, disease prevalence can sometimes be incalculable due to unavailability of samples. In these cases, the subtype disease prevalence can be substituted by the pooled disease prevalence estimate.

The results of the expression analysis can provide a statistical confidence level that a given diagnosis is correct. In some embodiments, such statistical confidence level can be above 85%, above 90%, above 91%, above 92%, above 93%, above 94%, above 95%, above 96%, above 97%, above 98%, above 99%, or above 99.5%.

### Subjects

In some embodiments, the present disclosure provides a system, method, or kit that includes or uses one or more subjects. In some embodiments, a subject is a biological entity containing expressed genetic materials. Examples of a biological entity include, but not limited to, a plant, animal, or microorganism, including, e.g., bacteria, viruses, fungi, and protozoa. In some embodiments, a subject includes tissues, cells, and progeny cells of a biological entity obtained in vivo or cultured in vitro.

In some embodiments, a subject is a mammal. In some embodiments, a subject is a human. In some embodiments, a human is a male or female. In additional embodiments, a human is from 1 day to about 1 year old, about 1 year old to about 3 years old, about 3 years old to about 12 years old, about 13 years old to about 19 years old, about 20 years old to about 40 years old, about 40 years old to about 65 years old, or over 65 years old.

In some embodiments, a subject is healthy or normal. In some embodiments, a subject is abnormal, or is diagnosed with, or suspected of being at a risk for, a disease. In some embodiments, a disease is a cancer, a disorder, a symptom, a syndrome, or any combination thereof.

### Samples

In some embodiments, the present disclosure provides a system, method, or kit that includes or uses one or more samples. The one or more samples used herein comprise any substance containing or presumed to contain nucleic acids. A sample can include a biological sample obtained from a subject. In some embodiments, a biological sample is a liquid sample. In some embodiments, a liquid sample is derived from whole blood, plasma, serum, ascites, cerebrospinal fluid, sweat, urine, tears, saliva, buccal sample, cavity rinse, or organ rinse. In some embodiments, a liquid sample is an essentially cell-free liquid sample or cell-free nucleic acid (cfNA). Non-limiting examples of cfNA include plasma, serum, sweat, plasma, urine, sweat, tears, saliva, sputum, and cerebrospinal fluid. For example, a sample can be cfDNA.

In some embodiments, a biological sample can include a solid biological sample, e.g., feces or tissue biopsy. In some embodiments, a sample can include in vitro cell culture constituents. Cell culture constituents can include, for example, conditioned medium from cell growth in a cell culture medium, recombinant cells, and cell components. In some embodiments, a sample can include a single cell, a cancer cell, a circulating tumor cell, a cancer stem cell, white blood cells, red blood cells, lymphocytes, and the like. In some embodiments, a sample can include a plurality of cells. In some embodiments, a sample can contain about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or 100% tumor cells. In some embodiments, a subject can be suspected to harbor a solid tumor or known to harbor a solid tumor. In some embodiments, a subject can have previously harbored a solid tumor.

A sample can be obtained invasively (e.g., a biopsy) or non-invasively (e.g., a swab or venipuncture). A biological sample can be obtained directly from a subject by, for example, accessing the circulatory system (e.g., intravenously or intra-arterially via a syringe), collecting a secreted biological sample (e.g., feces, urine, sputum, saliva), surgically extracting a sample (e.g., biopsy), swabbing (e.g., buccal swab, oropharyngeal swab), pipetting, and breathing. Moreover, a biological subject can be obtained from any anatomical part of a subject where a desired biological sample is located. Alternatively, a sample can be constructed by mixing biological and non-biological substances.

Samples can be obtained from the same subject at different time points. For example, a first sample can be collected from a diseased subject at a first time point and a second sample can be collected from the same diseased subject at a later time point. In some embodiments, a sample can be taken at a first time point and sequenced, and then another sample can be taken at a subsequent time point and sequenced.

Collecting and analyzing samples from the same subject at different time points may facilitate monitoring the progression of a disease or assessing the effectiveness of a treatment. In one example, a first sample can be collected from a diseased subject at a first time point and a second sample can be collected from the same subject at a later time point. These time points can be without treatment, or before and after treatment. In some embodiments, the two samples can allow determination of whether the disease has progressed or regressed. The data from the two time points also can be used to inform a treatment decision.

In some embodiments, the time between collections of samples from the same subject can be at least 1 hour, 2 hours, 4 hours, 6 hours, 8 hours, 12 hours, 24 hours, 48 hours, or more hours. Alternatively or in addition, the time between collection of samples from the same subject can be at least 1 day, 2 days, 4 days, 5 days, 7 days, 10 days, 2 weeks, 3 weeks, 4 weeks, 5 weeks, 6 weeks, 7 weeks, 8 weeks, 9 weeks, 10 weeks, 12 weeks, 15 weeks, 20 weeks, 25 weeks, 30 weeks, 40 weeks, 50 weeks, 1 year, or longer. The time between sample collections may vary for a given subject. For example, a sample can be collected at the commencement and completion of a treatment course, as well as one or more times during the treatment course. During treatment, a sample can be collected, for example, weekly or monthly. If a subject has entered a remission state, samples can be collected at regular intervals (e.g., monthly, biannually, or annually) to monitor the disease status of the subject.

A sample may have any suitable volume or quantity. For example, a sample may comprise at least about 1 nanoliter (nl), 2 nl, 5 nl, 10 nl, 20 nl, 50 nl, 100 nl, 200 nl, 500 nl, 1 microliter (μl), 2 μl, 5 μl, 10 μl, 20 μl, 25 μl, 50 μl, 100 μl, 200 μl, 300 μl, 400 μl, 500 μl, 600 μl, 700 μl, 800 μl, 900 μl, 1 milliliter (ml), 2 ml, 5 ml, 10 ml, 20 ml, 50 ml, 100 ml, or more than about 100 ml of a biological sample.

A sample may derive from a single source (e.g., a single subject or a single tissue or fluid sample) or multiple sources (e.g., multiple subjects or multiple tissues or fluid samples). For example, a sample can be a pooled sample, e.g., containing material from more than one organism, individual, or tissue.

A sample may comprise one or more nucleic acid molecules or fragments thereof. A nucleic acid molecule or fragment thereof can be separate from a cell (e.g., cell-free) or included within a cell. A nucleic acid molecule may comprise a nucleic acid fragment. A sample may comprise any useful amount of nucleic acid molecules or fragments thereof. For example, a sample may comprise a single nucleic acid molecule or fragment thereof or a collection of nucleic acid molecules or fragments thereof. A sample may comprise, for example, at least 1 femtogram (fg), 10 fg, 100 fg, 1 picogram (pg), 10 pg, 100 pg, 1 nanogram (ng), 10 ng, 50 ng, 100 ng, 500 ng, 1 microgram (μg), or more nucleic acid molecules or fragments thereof.

A nucleic acid molecule or fragment thereof may comprise a single strand or can be double-stranded. A sample may comprise one or more types of nucleic acid molecules or fragments thereof. Examples of nucleic acids include, but are not limited to, DNA, genomic DNA, plasmid DNA, cDNA, cfDNA, cell-free fetal DNA (cffDNA), circulating tumor DNA (ctDNA), nucleosomal DNA, chromatosomal DNA, mitochondrial DNA (miDNA), ribonucleic acid (RNA), messenger RNA (mRNA), transfer RNA (tRNA), micro RNA (miRNA), ribosomal RNA (rRNA), circulating RNA (cRNA), short hairpin RNA (shRNA), small interfering RNA (siRNA), an artificial nucleic acid analog, recombinant nucleic acid, plasmids, viral vectors, and chromatin. For example, a sample may comprise cfDNA.

cfDNA comprises non-encapsulated DNA in, e.g., a blood or plasma sample and can include ctDNA. cfDNA can be, for example, less than 200 base pairs (bp) long, such as between 120 and 180 bp long. These sequenced regions can be approximately 120-180 bp in size, which may reflect the size of nucleosomal DNA. Accordingly, a method of analyzing cfDNA, as disclosed herein, may facilitate the mapping of a nucleosome. Fragment pileups seen when cfDNA reads are mapped to a reference genome may reflect nucleosomal binding that protects certain regions from nuclease digestion during the process of cell death (apoptosis) or systemic clearance of circulating cfDNA by the liver and kidneys. A method of analyzing cfDNA can be complemented by, for example, digestion of a DNA or chromatin with MNase and subsequent sequencing (MNase sequencing). This method may reveal regions of DNA protected from MNase digestion due to binding of nucleosomal histones at regular intervals with intervening regions preferentially degraded, which reflects a footprint of nucleosomal positioning.

A nucleic acid molecule or fragment thereof may comprise one or more mutations. For example, a nucleic acid molecule or fragment thereof can include one or more insertions, deletions, and/or modifications. A mutation can be a somatic mutation or a germline mutation. A mutation can be associated with a disease such as a cancer. Examples of mutations include, but are not limited to, base substitutions, deletions (e.g., of a single base or base pair or a collection thereof), additions (e.g., of a single base or base pair or a collection thereof), duplications (e.g., of a single base or base pair or a collection thereof), copy number variations, gene fusions, transversions, translocations, inversions, indels, DNA lesions, aneuoploidy, polyploidy, chromosomal fusions, chromosomal structure alterations, chromosomal lesions, gene amplifications, gene duplications, gene truncations, and base modifications (e.g., methylation).

A nucleic acid molecule or fragment thereof may comprise any number of nucleotides. For example, a single-stranded nucleic acid molecule or fragment thereof may comprise at least 10, 20, 30, 40, 50, 60, 70, 80, 90, 100, 110, 120, 130, 140, 150, 160, 170, 180, 190, 200, 220, 240, 260, 280, 300, 350, 400, or more nucleotides. In the instance of a double-stranded nucleic acid molecule or fragment thereof, the nucleic acid molecule or fragment thereof may comprise at least 10, 20, 30, 40, 50, 60, 70, 80, 90, 100, 110, 120, 130, 140, 150, 160, 170, 180, 190, 200, 220, 240, 260, 280, 300, 350, 400, or more basepairs (bp), i.e. pairs of nucleotides. In some cases, a double-stranded nucleic acid molecule or fragment thereof may comprise between 100 and 200 bp, such as between 120 and 180 bp. For example, the sample may comprise a cfDNA molecule that comprises between 120 and 180 bp.

A sample comprising one or more nucleic acid molecules or fragments thereof can be processed to provide or purify a particular nucleic acid molecule or fragment thereof or collection thereof. For example, a sample comprising one or more types of nucleic acid molecules or fragments thereof (e.g., a combination of cfDNA and types of DNA or RNA) can be processed to separate one type of nucleic acid molecules or fragments thereof (e.g., cfDNA) from other types of nucleic acid molecules or fragments thereof. Alternatively, a sample comprising one or more nucleic acid molecules or fragments thereof of different sizes (e.g., lengths) can be processed to remove higher molecular weight and/or longer nucleic acid molecules or fragments thereof or lower molecular weight and/or shorter nucleic acid molecules or fragments thereof. Sample processing may comprise, centrifugation, filtration, selective precipitation, tagging, barcoding, partitioning, or any combination thereof. For example, cellular DNA can be separated from cell-free DNA by a selective polyethylene glycol and bead-based precipitation process, such as a centrifugation or filtration process. Cells included in a sample may or may not be lysed prior to separation of different types of nucleic acid molecules or fragments thereof. A processed sample may comprise, for example, at least 1 femtogram (fg), 10 fg, 100 fg, 1 picogram (pg), 10 pg, 100 pg, 1 nanogram (ng), 10 ng, 50 ng, 100 ng, 500 ng, 1 microgram (μg), or more of a particular size or type of nucleic acid molecules or fragments thereof

Materials and reagents useful for analyzing nucleic acids can be added to a sample. For example, a sample may comprise one or more buffers, salts, detergents, surfactants, stabilizers, denaturants, acids, bases, enzymes, oxidizers, barcodes, tags, unique molecular identifiers, fluorophores, dyes, primers, probes, or nucleotides. A sample may also comprise bisulfite ions. Examples of enzymes include polymerases (e.g., DNA or RNA polymerases), ligases, proteases, digestion enzymes, nucleases, and restriction enzymes. Nucleotides can include naturally occurring and/or non-naturally occurring nucleotides (e.g., modified nucleotides). For example, a nucleotide may comprise a nucleobase selected from the non-limiting group consisting of adenine, thymine, cytosine, uracil, guanine, xanthine, diaminopurine, deazaxanthine, deazaguanine, isocytosine, isoguanine, inosine, and modified versions thereof (e.g., by oxidation, reduction, and/or addition of a substituent such as an alkyl, hydroxyalkyl, hydroxyl, or halogen moiety). A nucleotide may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide may also comprise a modified linker moiety (e.g., in lieu of a phosphate moiety). A nucleotide can include a detectable moiety such as a fluorescent tag.

Materials and reagents can be added to the sample at any time. For example, a material or reagent can be added to the sample prior to sample processing (e.g., isolation or extraction of a particular size or type of nucleic acid molecules or nucleic acid fragments), prior to processing (e.g., modification) of nucleic acid molecules or nucleic acid fragments, prior to sequencing of a nucleic acid molecule or fragment thereof, or at any other time. In some cases, different materials and reagents can be added at different times during analysis of a sample. For example, a reagent suitable for stabilizing a sample or a component thereof can be added immediately after collection of a sample and prior to any processing or analysis, and reagents for analyzing a nucleic acid molecule or fragment thereof can be added at a later point in time.

In some embodiments, the present disclosure provides a method to diagnose a cancer. A sample can be derived from a subject that is healthy or believed to be healthy, suspected or having a disease, known to have a disease, or known to have previously had a disease. A disease can be a cancer or neoplasia. A cancer can be, for example, blastoma, carcinoma, lymphoma, leukemia, sarcoma, seminoma, or dysgerminoma. Non-limiting examples of cancers that can be inferred by the disclosed methods include acute lymphoblastic leukemia (ALL), acute myeloid leukemia (AML), adrenocortical carcinoma, AIDS-related lymphoma, anal cancer, astrocytoma, atypical teratoid/rhabdoid tumor, basal cell carcinoma, bile duct cancer, bladder cancer, bone cancer, Ewing sarcoma, osteosarcoma, malignant fibrous histiocytoma, brain tumors, brain cancer, breast cancer, bronchia tumors, Burkitt lymphoma, Non-Hodgkin's lymphoma, Kaposi sarcoma, carcinoid tumor (gastrointestinal), cardiac (heart) tumors, embryonal tumors, germ cell tumor, primary central nervous system (CNS) lymphoma, cervical cancer, cholangiocarcinoma, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, ductal carcinoma in situ (DCIS), endometrial cancer, ependymoblastoma, ependymoma, esophageal cancer, esthesioneuroblastoma, extracranial germ cell tumor, medulloblastoma, medulloeptithelioma, extragonadal germ cell tumor, eye cancer, intraocular melanoma, retinoblastoma, fallopian tube cancer, gallbladder cancer, gastric (stomach) cancer, gastrointestinal carcinoid tumor, gastrointestinal stromal tumors (GIST), soft tissue sarcoma, germ cell tumors, extracranial germ cell tumors, extragonadal germ cell tumors, ovarian germ cell tumors, testicular cancer, gestational trophoblastic disease, hairy cell leukemia, head and neck cancer, hypopharyngeal cancer, laryngeal cancer, heart tumors, hepatocellular (liver) cancer, Langerhans cell histiocytosis, Hodgkin's lymphoma, intraocular melanoma, islet cell tumors, pancreatic neuroendocrine tumors, kidney (renal cell) cancer, papillomatosis, leukemia, lip and oral cavity cancer, liver cancer, lung cancer (non-small cell and small cell), lymphoma, melanoma, Merkel cell carcinoma, skin cancer, mesothelioma, metastatic cancer, metastatic squamous neck cancer with occult primary, midline tract carcinoma involving nut gene, mouth cancer, multiple endocrine neoplasia syndromes, multiple myeloma/plasma cell neoplasms, mycosis fungoides, myelodysplastic syndromes, myelodysplastic/myeloproliferative neoplasms, chronic myeloproliferative neoplasms, nasal cavity and paranasal sinus cancer, nasopharyngeal cancer, neuroblastoma, oral cancer, lip and oral cavity cancer, oropharyngeal cancer, ovarian cancer, pancreatic cancer, paraganglioma, parathyroid cancer, penile cancer, pharyngeal cancer, pheochromocytoma, pituitary tumor, pleuropulmonary blastoma, primary peritoneal cancer, prostate cancer, rectal cancer, recurrent cancer, rhabdomyosarcoma, salivary gland cancer, sarcoma, vascular tumors, uterine sarcoma, Sézary syndrome, small intestine cancer, squamous cell carcinoma of the skin, diffuse B-cell lymphoma, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, carcinoma of unknown primary, urethral cancer, uterine cancer, uterine sarcoma, vaginal cancer, vulvar cancer, Waldenstrom macroglobulinemia, and Wilms tumor. In some cases, a subject may have a benign tumor.

### Colorectal Cancer

The present disclosure provides a method to diagnose colorectal cancer. Most colorectal cancers develop from polyps, which are abnormal growths inside the colon or rectum. Colorectal adenomas are precursor lesions of colorectal carcinoma. Advanced adenoma can be defined as a subset of adenoma in which the lesion size measures 10 mm or more and contains a substantially villous component or high grade dysplasia. Only about 1-10% of people with adenomas develop colorectal carcinoma, while significantly more advanced adenoma patients eventually advance to colorectal carcinoma. Thus, early detection and removal of advanced adenomas can dramatically decrease the incidence of colorectal carcinoma. Samples obtained from polyps or adenomas can be used to diagnose colorectal cancer.

### Nucleic Acids

In some embodiments, the present disclosure provides a system, method, or kit that analyzes nucleic acids. Analysis of nucleic acid molecules can involve providing a sample comprising a nucleic acid molecule and subjecting the nucleic acid molecule to conditions sufficient to modify the nucleic acid molecule. The modified nucleic acid molecule can be sequenced (e.g., using next generation sequencing techniques) to generate sequence reads, which can be used to determine a genetic sequence feature, for example, by measuring gene expression levels or determining an expression profile.

In some embodiments, nucleic acids containing germline sequences can be extracted from a biological sample of a subject. In some embodiments, the biological sample is a solid tissue. The biological sample can be tissue, such as normal or healthy tissue from the subject. The biological sample can be a liquid sample, including, for example, blood, buffy coat from blood (which can include lymphocytes), saliva, or plasma.

In some embodiments, nucleic acids that contain somatic variants can be extracted from a biological sample of a subject. In some embodiments, a biological sample can include a solid tissue, a primary tumor, a metastasis tumor, a polyp, or an adenoma. In some embodiments, a biological sample can include a liquid sample, urine, saliva, cerebrospinal fluid, plasma, or serum. In some embodiments, the liquid is a cell-free liquid. In some embodiments, cells from a liquid sample can be enriched or isolated. In some embodiments, the sample can include cell-free nucleic acid, e.g., DNA or RNA. In some embodiments, nucleic acids described herein can include RNA, DNA, genomic DNA, mitochondrial DNA, viral DNA, synthetic DNA, or cDNA reverse transcribed from RNA.

Modifying a nucleic acid molecule can include degradation or fragmentation of the nucleic acid molecule. The degree of degradation or fragmentation can be estimated using, for example, gel-based electrophoresis, mass spectrometry, high performance liquid chromatography (HPLC), quantitative PCR (qPCR), and/or droplet digital PCR. A portion of a sample (e.g., one or more nucleic acid molecules or fragments thereof) can be reserved for such an analysis, or a separate sample can be used to perform such an analysis. Performing a gel-based electrophoretic analysis may comprise, for example, loading a sample including nucleic acid molecules or fragments thereof onto a gel (e.g., a PAGE, agarose or other molecular sieve gel) which may or may not contain an embedded fluorescent DNA stain, performing electrophoresis, staining the gel if necessary, and detecting fluorescence. A densitometry analysis may also be performed. A mass spectrometric, HPLC, or qPCR analysis can be similarly used to determine the degree of degradation or fragmentation that can be expected in analyses of future samples. Sample loss following nucleic acid molecule modification (e.g., bisulfite conversion) can be minimized by optimizing reaction conditions such as the bisulfite concentration, exposure time to bisulfite, the conversion temperature, pH, and inclusion of chemical protectants.

The present disclosure provides methods for determining a genetic sequence feature. The genetic sequence feature can be determined based on sequence reads or degradation parameters. A genetic sequence feature can be a methylation status of a nucleic acid molecule or fragment thereof, a single nucleotide polymorphism, a copy number variation, an indel, and a structural variant. A genetic sequence feature can be useful for diagnosing a subject with a disease, or monitoring progression of a disease. For example, the disease may be a cancer and a genetic sequence feature can be used for identifying the cancer's tissue-of-origin and estimating tumor burden.

Nucleic acid molecules can be extracted from biological samples by contacting the biological samples with an array of probes under conditions to allow hybridization. The degree of hybridization may be assayed in a quantitative matter using methods known in the art. In some cases, the degree of hybridization at a probe position may be related to the intensity of signal provided by the assay, which therefore is related to the amount of complementary nucleic acid sequence present in the sample. Computer-implemented software can be used to extract, normalize, summarize, and analyze array intensity data from probes across the human genome or transcriptome including expressed genes, exons, introns, and miRNAs. In some embodiments, the intensity of a given probe in either the benign or malignant samples can be compared against a reference set to determine whether differential expression is occurring in a sample. An increase or decrease in relative intensity at a marker position on an array corresponding to an expressed sequence is indicative of an increase or decrease respectively of expression of the corresponding expressed sequence. Alternatively, a decrease in relative intensity may be indicative of a mutation in the expressed sequence.

The resulting intensity values for each sample can be analyzed using feature selection techniques including filter techniques which assess the relevance of features by looking at the intrinsic properties of the data, wrapper methods which embed the model hypothesis within a feature subset search, and embedded techniques in which the search for an optimal set of features is built into a classifier algorithm.

Filter techniques useful for the methods disclosed herein include (1) parametric methods, such as the use of two sample t-tests, ANOVA analyses, Bayesian frameworks, and Gamma distribution models; (2) model free methods, such as the use of Wilcoxon rank sum tests, between-within class sum of squares tests, rank products methods, random permutation methods, or TNoM which involves setting a threshold point for-fold-change differences in expression between two datasets and then detecting the threshold point in each gene that minimizes the number of misclassifications; and (3) multivariate methods, such as bivariate methods, correlation based feature selection methods (CFS), minimum redundancy maximum relevance methods (MRMR), Markov blanket filter methods, and uncorrelated shrunken centroid methods. Wrapper methods useful in the methods of the present disclosure include sequential search methods, genetic algorithms, and estimation of distribution algorithms. Embedded methods useful in the methods of the present disclosure include random forest algorithms, weight vector of support vector machine algorithms, and weights of logistic regression algorithms.

Selected features may then be classified using a classifier algorithm. Illustrative algorithms include, but are not limited to, methods that reduce the number of variables, such as principal component analysis algorithms, partial least squares methods, and independent component analysis algorithms. Illustrative algorithms further include but are not limited to methods that handle large numbers of variables directly, such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof.

### Data Analysis Overview

In some embodiments, the present disclosure provides a system, method, or kit that can include data analysis realized in software application, computing hardware, or both. An analysis application or system can include at least a data receiving module, a data pre-processing module, a data analysis module (which can operate on one or more types of genomic data), a data interpretation module, or a data visualization module. A data receiving module can comprise computer systems that connect laboratory hardware or instrumentation with computer systems that process laboratory data. A data pre-processing module can comprise hardware systems or computer software that performs operations on the data in preparation for analysis. Examples of operations that can be applied to the data in the pre-processing module include affine transformations, denoising operations, data cleaning, reformatting, or subsampling. A data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In some embodiments, the methods disclosed herein can include computational analysis on nucleic acid sequencing data of samples from an individual or from a plurality of individuals. An analysis can identify a variant inferred from sequence data to identify sequence variants based on probabilistic modeling, statistical modeling, mechanistic modeling, network modeling, or statistical inferences. Non-limiting examples of analysis methods include principal component analysis, autoencoders, singular value decomposition, Fourier bases, wavelets, discriminant analysis, regression, support vector machines, tree-based methods, networks (e.g., neural networks), matrix factorization, and clustering. Non-limiting examples of variants include a germline variation or a somatic mutation. In some embodiments, a variant can refer to an already-known variant. The already-known variant can be scientifically confirmed or reported in literature. In some embodiments, a variant can refer to a putative variant associated with a biological change. A biological change can be known or unknown. In some embodiments, a putative variant can be reported in literature, but not yet biologically confirmed. Alternatively, a putative variant is never reported in literature, but can be inferred based on a computational analysis disclosed herein. In some embodiments, germline variants can refer to nucleic acids that induce natural or normal variations.

Natural or normal variations can include, for example, skin color, hair color, and normal weight. In some embodiments, somatic mutations can refer to nucleic acids that induce acquired or abnormal variations. Acquired or abnormal variations can include, for example, cancer, obesity, conditions, symptoms, diseases, and disorders. In some embodiments, the analysis can include distinguishing between germline variants. Germline variants can include, for example, private variants and somatic mutations. In some embodiments, the identified variants can be used by clinicians or other health professionals to improve health care methodologies, accuracy of diagnoses, and cost reduction.

Provided herein are improved methods and computing systems or software media that can distinguish among sequence errors in nucleic acid introduced through amplification and/or sequencing techniques, somatic mutations, and germline variants. Methods provided can include simultaneously calling and scoring variants from aligned sequencing data of all samples obtained from a subject. Samples obtained from subjects other than the subject can also be used. Other samples can also be collected from subjects previously analyzed by a sequencing assay or a targeted sequencing assay (i.e. a targeted resequencing assay). Methods, computing systems, or software media disclosed herein can improve identification and accuracy of variations or mutations (e.g., germline or somatic, including copy number variations, single nucleotide variations, indels, a gene fusions), and lower limits of detection by reducing the number of false positive and false negative identifications.

Processing a nucleic acid molecule or fragment thereof may comprise performing nucleic acid amplification. For example, any type of nucleic acid amplification reaction can be used to amplify a target nucleic acid molecule or a fragment thereof to generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Non-limiting examples of PCR include quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification can be isothermal or may comprise thermal cycling. Thermal cycling may comprise two or more discrete temperature steps. A temperature step may be associated with a particular process, such as initialization, denaturation, annealing, and extension. A single thermal cycle may include denaturation, annealing, and extension. Multiple thermal cycles can be performed to amplify a nucleic acid molecule or fragment thereof to a detectable level.

### Global Dynamic Downsampling

In some embodiments, the present disclosure provides a system, method, or kit that can include global dynamic downsampling. In some embodiments, global dynamic downsampling can be used for subject background imputation. In some embodiments, changes detected in sequences can be germline variations that are discordant with the reference genome. In other words, genetic profiles of an individual can be different from genetic profiles of a canonical human genome and not the causative somatic mutations that are associated with age-associated diseases. In some embodiments, filtering out germline variations can be based on sequencing the subject-matched background genomic information. For example, DNA of leukocyte white blood cells, which would be normal healthy subject background in the absence of leukemia can be filtered out.

In some embodiments, the majority of cfDNA collected from an individual, even with an advanced disease state, is not from aberrant cells. In such embodiments, stochastically downsampling the sequence data can be used to enrich the aberrant cells. In some embodiments, one or more reads can be removed from the aberrant cells to filter out the germline variations by comparing the downsampled sequence data to the reference genome.

To ensure that an arbitrary fraction of reads is not removed in the downsampling, the process can begin with analyzing a potential depth of mutational “signal” reads by calculating the fraction of reads <10% that show a different base (or insertion or deletion) than what the majority of the reads (>90%) show. This fraction can be calculated over each window (size>=1 bp) across the genome to calculate a weighted average, minimum and maximum fractions. In some embodiments, a fraction calculation of a particular window can be normalized to the number of reads, but also weighted by the number of reads such that the greater the number of reads covering a window, the more weight is given to the ratio calculated within that window to the overall average. This process assumes that areas of the genome covered by more reads can give a more accurate fraction than the areas with less coverage.

In some embodiments, once a weighted average has been calculated, the data analysis stochastically can remove reads until the weighted average ratio of reads can be removed globally. In some embodiments, this removal can be designed on a per-window basis. In some embodiments, the data analysis can perform the stochastic removal several times (10-100) independently to make sure that the proper downsampling is performed. In some embodiments, removal of reads can occur recursively.

In some embodiments, final analysis can include independent runs of downsampled datasets being mapped against the reference human genome (hg19) and compared. Where the sequences of the majority of independent runs differ from the reference, the reference sequence can be overridden. In areas where the sequence coverage of downsampled datasets are insufficient (e.g., <3 reads), the analysis can retain the reference sequence. Ultimately, the analysis can achieve construction of a subject-matched healthy reference to compare against for the rest of the analysis.

### Biological Conditions

In some embodiments, the present disclosure provides a system, method, or kit that can include a first and a second sample collected from a same subject at different biological conditions. In some embodiments, the system, media, method, or kit disclosed herein can include evaluating or predicting a biological condition. In some embodiments, the system, media, method, or kit disclosed herein can include evaluating or predicting a state or condition. The state or condition can be past, present, or future.

In some embodiments, a biological condition can include a disease. In some embodiments, a biological condition can be a stage of a disease. In some embodiments, a biological condition can be an age-associated disease. In some embodiments, a biological condition can be aging. In some embodiments, a biological condition can be a state in aging. In some embodiments, a biological condition can be a gradual change of a biological state. In some embodiments, a biological condition can be a treatment effect. In some embodiments, a biological condition can be a drug effect. In some embodiments, a biological condition can be a surgical effect. In some embodiments, a biological condition can be a biological state after a lifestyle modification. Non-limiting examples of lifestyle modifications include a diet change, a smoking change, and a sleeping pattern change.

In some embodiments, a biological condition is unknown. The analysis described herein can include machine learning to infer an unknown biological condition or to interpret the unknown biological condition.

### Risk States

In some embodiments, the present disclosure provides a system, method, or kit that includes a first sample and a second sample collected from a subject that differ by risk for developing a biological condition. In some embodiments, the system, media, method, or kit disclosed herein can include evaluating or predicting a risk state.

In some embodiments, a risk state can include the risk for developing a disease state. In some embodiments, a risk state can be a stage of a disease. In some embodiments, the risk state can be an age-associated disease. In some embodiments, a risk state can include one or more aspects associated with aging. In some embodiments, a risk state can be a state in aging. In some embodiments, a risk state can be a treatment effect, side effect, or non-intended impact of medical treatment. In some embodiments, a risk state can be a surgical outcome. In some embodiments, a risk effect can be a biological state that can occur after a lifestyle modification. Non-limiting examples of lifestyle modifications include a diet change, a smoking change, and a sleeping pattern change.

In some embodiments, a risk state is unknown. The present disclosure provides a system, method, or kit that can include machine learning to infer an unknown risk state or to interpret the unknown risk state.

### Digital Processing Device

In some embodiments, the subject matter described herein can include a digital processing device, or use of the same. In some embodiments, the digital processing device can include one or more hardware central processing units (CPU), graphics processing units (GPU), or tensor processing units (TPU) that carry out the device's functions. In some embodiments, the digital processing device can include an operating system configured to perform executable instructions. In some embodiments, the digital processing device can optionally be connected a computer network. In some embodiments, the digital processing device can be optionally connected to the Internet such that it accesses the World Wide Web. In some embodiments, the digital processing device can be optionally connected to a cloud computing infrastructure. In some embodiments, the digital processing device can be optionally connected to an intranet. In some embodiments, the digital processing device can be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can include, for example, those with booklet, slate, and convertible configurations known to those having ordinary skill in the art.

In some embodiments, the digital processing device can include an operating system configured to perform executable instructions. For example, the operating system can include software, including programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some embodiments, the operating system can be provided by cloud computing, and cloud computing resources can be provided by one or more service providers.

In some embodiments, the device can include a storage and/or memory device. The storage and/or memory device can be one or more physical apparatuses used to store data or programs on a temporary or permanent basis. In some embodiments, the device can be volatile memory and require power to maintain stored information. In some embodiments, the device can be non-volatile memory and retain stored information when the digital processing device is not powered. In some embodiments, the non-volatile memory can include flash memory. In some embodiments, the non-volatile memory can include dynamic random-access memory (DRAM). In some embodiments, the non-volatile memory can include ferroelectric random access memory (FRAM). In some embodiments, the non-volatile memory can include phase-change random access memory (PRAM). In some embodiments, the device can be a storage device including, for example, CD-ROMs, DVDs, flash memory devices, magnetic disk drives, magnetic tapes drives, optical disk drives, and cloud computing-based storage. In some embodiments, the storage and/or memory device can be a combination of devices such as those disclosed herein.

In some embodiments, the digital processing device can include a display to send visual information to a user. In some embodiments, the display can be a cathode ray tube (CRT). In some embodiments, the display can be a liquid crystal display (LCD). In some embodiments, the display can be a thin film transistor liquid crystal display (TFT-LCD). In some embodiments, the display can be an organic light emitting diode (OLED) display. In some embodiments, on OLED display can be a passive-matrix OLED (PMOLED) or active-matrix OLED (AMOLED) display. In some embodiments, the display can be a plasma display. In some embodiments, the display can be a video projector. In some embodiments, the display can be a combination of devices such as those disclosed herein.

In some embodiments, the digital processing device can include an input device to receive information from a user. In some embodiments, the input device can be a keyboard. In some embodiments, the input device can be a pointing device including, for example, a mouse, trackball, track pad, joystick, game controller, or stylus. In some embodiments, the input device can be a touch screen or a multi-touch screen. In some embodiments, the input device can be a microphone to capture voice or other sound input. In some embodiments, the input device can be a video camera to capture motion or visual input. In some embodiments, the input device can be a combination of devices such as those disclosed herein.

### Non-Transitory Computer-Readable Storage Medium

In some embodiments, the subject matter disclosed herein can include one or more non-transitory computer-readable storage media encoded with a program including instructions executable by the operating system of an optionally networked digital processing device. In some embodiments, a computer-readable storage medium can be a tangible component of a digital processing device. In some embodiments, a computer-readable storage medium can be optionally removable from a digital processing device. In some embodiments, a computer-readable storage medium can include, for example, CD-ROMs, DVDs, flash memory devices, solid state memory, magnetic disk drives, magnetic tape drives, optical disk drives, cloud computing systems and services, and the like. In some embodiments, the program and instructions can be permanently, substantially permanently, semi-permanently, or non-transitorily encoded on the media.

### Computer Systems

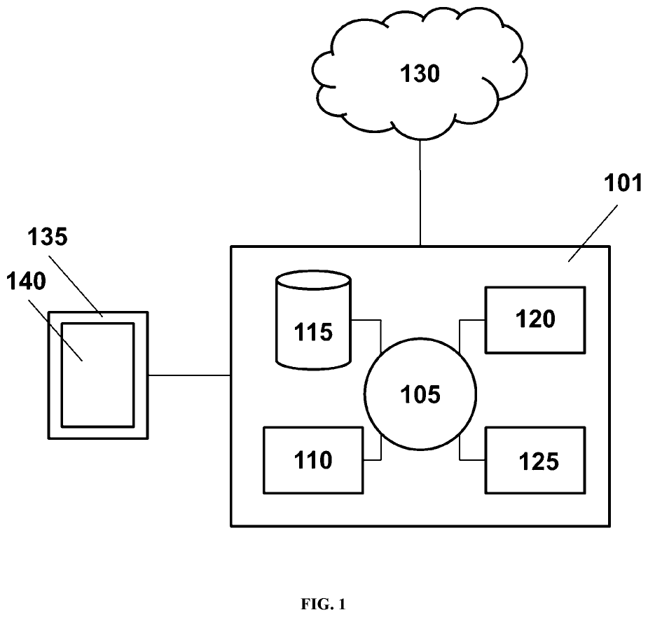

The present disclosure provides computer systems that are programmed to implement methods of the disclosure. FIG. 1 shows a computer system 101 that is programmed or otherwise configured to store, process, identify, or interpret subject data, biological data, biological sequences, or reference sequences. The computer system 101 can process various aspects of subject data, biological data, biological sequences, or reference sequences of the present disclosure, such as, for example, DNA regulatory elements and/or RNA regulatory elements. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some embodiments is a telecommunication and/or data network. The network 130 can include one or more computer servers, which can enable distributed computing, such as cloud computing. The network 130, in some embodiments with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries and saved programs. The storage unit 115 can store user data, e.g., user preferences and user programs. The computer system 101 in some embodiments can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some embodiments, the code can be retrieved from the storage unit 115 and stored on the memory 110 for ready access by the processor 105. In some embodiments, the electronic storage unit 115 can be precluded, and machine-executable instructions are stored on memory 110.

The code can be pre-compiled and configured for use with a machine having a processer adapted to execute the code, or can be interpreted or compiled during runtime. The code can be supplied in a programming language that can be selected to enable the code to execute in a pre-compiled, interpreted, or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, a nucleic acid sequence, an enriched nucleic acid sample, an expression profile, and an analysis of an expression profile. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, probe a plurality of regulatory elements, sequence a nucleic acid sample, enrich a nucleic acid sample, determine an expression profile of a nucleic acid sample, analyze an expression profile of a nucleic acid sample, and archive or disseminate results of analysis of an expression profile.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

In some embodiments, the subject matter disclosed herein can include at least one computer program, or use of the same. A computer program can a sequence of instructions, executable in the digital processing device's CPU, GPU, or TPU, written to perform a specified task. Computer-readable instructions can be implemented as program modules, such as functions, objects, Application Programming Interfaces (APIs), data structures, and the like, that perform particular tasks or implement particular abstract data types. In light of the disclosure provided herein, those having ordinary skill in the art will recognize that a computer program can be written in various versions of various languages.

The functionality of the computer-readable instructions can be combined or distributed as desired in various environments. In some embodiments, a computer program can include one sequence of instructions. In some embodiments, a computer program can include a plurality of sequences of instructions. In some embodiments, a computer program can be provided from one location. In some embodiments, a computer program can be provided from a plurality of locations. In some embodiments, a computer program can include one or more software modules. In some embodiments, a computer program can include, in part or in whole, one or more web applications, one or more mobile applications, one or more standalone applications, one or more web browser plug-ins, extensions, add-ins, or add-ons, or combinations thereof.

In some embodiments, the computer processing can be a method of statistics, mathematics, biology, or any combination thereof. In some embodiments, the computer processing method includes a dimension reduction method including, for example, principal component analysis, autoencoders, singular value decomposition, Fourier bases, wavelets, or discriminant analysis.

In some embodiments, the computer processing method is a supervised machine learning method including, for example, regressions, support vector machines, tree-based methods, neural networks, and nearest neighbor methods.

In some embodiments, the computer processing method is an unsupervised machine learning method including, for example, clustering, neural networks, principal component analysis, and matrix factorization.

### Databases

In some embodiments, the subject matter disclosed herein can include one or more databases, or use of the same to store subject data, biological data, biological sequences, or reference sequences. Reference sequences can be derived from a database. Reference sequences can be obtained from a subject. The subject can be a healthy subject or a subject suspected to have or has a disease, e.g, a cancer. Reference sequences can also be obtained from an artificial sequence. In view of the disclosure provided herein, those having ordinary skill in the art will recognize that many databases can be suitable for storage and retrieval of the sequence information. In some embodiments, suitable databases can include, for example, relational databases, non-relational databases, object oriented databases, object databases, entity-relationship model databases, associative databases, and XML databases. In some embodiments, a database can be internet-based. In some embodiments, a database can be web-based. In some embodiments, a database can be cloud computing-based. In some embodiments, a database can be based on one or more local computer storage devices.

### EXAMPLES

### Example 1

### Transcriptional Start Site (TSS) Panel

Data files defining the locations of TSSs and expressed enhancers were obtained from the FANTOMS (Functional ANnoTation Of the Mammalian genome) project phase 2.2 cap analysis gene expression (CAGE) peak liftover data. The reference human genome (hg19) was mapped to the newer reference human genome (hg38). The “problematic” or non-liftover peaks were omitted. Because FANTOMS does not provide an hg38 mapping of enhancer sites, hg19-mapped enhancer sites were used instead. UCSC liftOver was used to remap from the “Feb 2009 (GRCh37/hg19)” assembly to the “Dec 2013 (GRCh38/hg38)” assembly with the following default parameters: minimum ratio of bases that must remap=0.95; allow multiple output regions=FALSE; minimum hit size in query=0; minimum chain size in target=0; minimum ratio of alignment blocks or exons that must map=1; and if thickStart/thickEnd is not mapped, use the closest mapped base=FALSE. The loci that failed liftOver were excluded from the analysis. The successful (correct) liftOver loci were identified as human permissive enhancers of hg38 liftover.

### Analysis Windows

Each cluster was systemically expanded by varying fixed amounts around either the cluster midpoint or the position of the maximum-score CAGE peak. Windows were grown by 2-7 nucelosome sizes upstream and 1-6 nucleosomes downstream (1 nucleosome=170 bp). The size of the resulting capture regions of interest (ROIs) were computed by taking the union of all resulting intervals.

Clustering window has a small effect on overall ROI size because most analysis windows are large enough to cover the cluster windows. Accordingly, we designed the ROI at the smallest clustering window to allow for analytical flexibility downstream. At the smallest clustering window, midpoint vs maximum CAGE score makes almost no difference to the ROI. Thus, either method does not affect capture panel design.

For a computational analysis with midpoint design, a 100 bp cluster window was used in the FANTOM analysis. To reduce the number of putative transcription start sites to a tractable number, clustering was used. In short, starting at position 1 on each chromosome and sweeping to the right, if a peak was within 100 bp of the peak nearest to its left, it was moved into the same cluster, and then either the midpoint of the cluster or the position of the peak with the highest CAGE score was used as a TSS. It also is possible to cluster based on maximum distance rather than closes distance, in which case a peak is joined to a cluster if it is within 100 bp of the furthest peak in that cluster.
The window size used was −510/+510 bp.

### Sequencing Bandwidth

Sequence capacity was as follows:
NextSeq=−400-600 Mbp fragments (SE reads)/flowcell
Average fragment length=−170 bp
   Taking into account some off-targeting and duplication, the sequencing bandwidth parameters are shown in TABLE 1 below:

TABLE 1

| | |
|---|---|
| Fragment length | 170 |
| Frags/Mb | 5882.352941 |
| Frags/Mb @ 30x | 176470.5882 |
| On-target rate | 0.8 |
| Duplication rate | 0.1 |
| Effective frags/Mb @ 30x | 245098.0392 |

| | # of fragments/FC | | |
|---|---|---|---|
| Panel size (Mb) | 400,000,000.00 | 500,000,000.00 | 600,000,000.00 |
| 50 | 32 samples/FC | 40 | 48 |
| 70 | 23 | 29 | 34 |
| 88 | 18 | 23 | 27 |
| 102 | 16 | 20 | 24 |
| 120 | 13 | 17 | 20 |

The computational analysis resulted in a TSS panel for use in a whole promoter sequencing (WPS) method, as shown in TABLE 2, incorporated herein in its entirety. TABLE 2 illustrates an example panel showing resulting loci of TSS after enrichment with a probe set of the present disclosure. The REGION NAME or TSS region name is the FANTOMS name from hg19 coordinates of the input BED file(s) or the default name of the selection region. The region name takes the format of CHROMOSOME: START-STOP. The start and stop locations are the start and stop region coordinates, respectively. The region length is the number of bases in the region, which can be calculated by the difference between the start and stop locations.

For each probe, various parameters can be calculated. Parameters can include, for example, any of the following:

Bases probe coverage: the number of bases in the region which are directly covered by a capture probe. For example, the values can vary from 0 to about 20,000.

Fractional probe coverage: the fractional percentage of bases which are directly covered by a capture probe. For example, a value of 1.000 means 100% coverage, where every base of the target is covered by one or more capture probes. A value of 0.460 means that 46% of the region is covered by one or more capture probes. For example, the values can vary from 0 to 1.

Bases-estimated probe coverage: the number of bases in the region directly covered by a probe or by indirect/adjacent coverage. The base-estimated probe coverage is an estimate of the actual amount of sequence that be captured by a capture probe, determined from empirical tests predicting that capture probes can hybridize to the end of library insert and extend coverage away from the probe. The 100 bp capture padding was validated with Illumina dual-end sequencing, using a typical library size of −200 bp. This number may not be accurate for libraries with much larger or smaller insert sizes, or single end reads. For example, the values can vary from 0 to about 20,000.

Fractional bases-estimated probe coverage: the percent coverage of the region, as a fraction of 1, using indirect/adjacent coverage. For example, a value 0.982 means that 98.2% of the target is covered indirectly by one or more capture probes. For example, the values can vary from 0 to 1.

Bases without probe coverage: the number of bases in the region that are not directly covered by a capture probe. For example, bases-estimated without probe coverage can vary from 0 to about 5,000.

Predicted bases without probe coverage: the number of bases in the region that are not covered indirectly and are likely to be missed during capture. For example, the values can vary from 0 to about 5,000.

Bases without probe coverage due to N: the number of bases in the region that are not covered directly by probes due to the region containing N's or ambiguous bases in the source. For example, the values can vary from 0 to about 1,000.

Bases without probe coverage due to repeats: the number of bases in the region that are not covered directly by probes due to the region containing low complexity or highly repetitive sequence. For example, the values can vary from 0 to about 3,000.

Bases-estimated without probe coverage: the number of bases in the region not directly covered by a probe or by indirect/adjacent coverage. For example, the values can vary from 0 to 3,000.

Bases-estimated without probe coverage due to N: the number of bases in the region that are not covered indirectly due to the region containing N's or ambiguous bases in the source. For example, the values can vary from 0 to about 1,000.

Bases-estimated without probe coverage due to repeats: the number of bases in the region that are not covered indirectly due to the region containing repetitive sequence. For example, the values can vary from 0 to about 3,000.

### Example 2

### Diagnosing Cancer by Analysis of TSS Expression Profile

A nucleic acid test sample is collected from a human subject and purified . The purified nucleic acid test sample is then be enriched using a probe set containing hybridization probes having sequence complementarity to TSS loci identified by a reference database. The enriched nucleic acid sequence is optionally amplified using barcoding methods and a sequencing library is prepared. The amplified and enriched nucleic acids are then loaded onto a sequencer to obtain sequence reads.

The sequence reads are then analyzed by computer-implemented statistical and mathematical methods to generate a TSS expression profile, which identifies TSS availability for the test sample. TSS availability is determined by quantifying the sequencing reads of the TSS loci, i.e. the greater number of sequencing reads suggests greater availability of the TSS. Gene

The resulting TSS profile obtained from the test sample is then compared to control TSS expression profiles for “healthy” and “disease” (e.g., cancer) states using statistical methods. Healthy and diseases profiles can be obtained by sequencing samples from subjects having the disease and not having the disease, or from a reference database.

While preferred embodiments have been shown and described herein, it will be obvious to those having ordinary skill in the art that such embodiments are provided by way of example only. Numerous variations, changes, and substitutions will now occur to those having ordinary skill in the art without departing from the invention. It should be understood that various alternatives to the embodiments described herein can be employed in practicing the disclosure. It is intended that the following claims define the scope of the invention and that methods and structures within the scope of these claims and their equivalents be covered thereby.

1-74. (canceled)

75. A method for processing a nucleic acid sample of a subject, comprising:
(a) using a probe set comprising probes having sequence complementarity with a plurality of regulatory elements to enrich for nucleic acid sequences in said nucleic acid sample, wherein said nucleic acid sequences comprise at least a subset of said plurality of regulatory elements, thereby providing an enriched nucleic acid sample;
(b) directing said enriched nucleic acid sample or a derivative thereof to nucleic acid sequencing to generate a plurality of sequence reads comprising sequences that align with said subset of said plurality of regulatory elements;
(c) computer processing said plurality of sequence reads to determine an expression profile of genes operably linked to said subset of said plurality of regulatory elements; and
(d) using said expression profile of genes to identify a disease in said subject at an accuracy of at least 90%.

76. The method of claim 75, wherein said regulatory elements are transcriptional start sites (TSS), enhancer sites, silencers, promoters, operators, untranslated regions (UTR), leader sequences (5′ UTR), trailer sequences (3′ UTR), terminators, or any combination thereof.

77. The method of claim 75, further comprising, prior to (b), processing said nucleic acid sample with a plurality of barcodes.

78. The method of claim 77, wherein said plurality of barcodes comprises unique molecular identifiers.

79. The method of claim 75, wherein said regulatory elements are microRNA (miRNA) regulatory elements, messenger RNA (mRNA) regulatory elements, small interfering RNA regulatory elements, (siRNA) regulatory elements, piwi-interacting RNA (piRNA) regulatory elements, small nucleolar RNA (snoRNA) regulatory elements, small nuclear RNA (snRNA) regulatory elements, extracellular RNA (exRNA) regulatory elements, small Cajal body-specific RNA (scaRNA) regulatory elements, non-coding RNA (ncRNA) regulatory elements, or any combination thereof.

80. The method of claim 75, wherein said computer processing of said plurality of sequence reads is using statistics, mathematics, or biology.

81. The method of claim 75, wherein said computer processing of said plurality of sequence reads is a dimension reduction method.

82. The method of claim 81, wherein said dimension reduction method is principal component analysis, autoencoding, singular value decomposition, Fourier bases, wavelets, or discriminant analysis.

83. The method of claim 75, wherein said computer processing of said plurality of sequence reads comprises a supervised machine learning method, wherein said supervised machine learning method is a regression, support vector machine, tree-based method, neural network, or nearest neighbor method.

84. The method of claim 75, wherein said computer processing method comprises an unsupervised machine learning method, wherein said unsupervised machine learning method is clustering, neural network, principal component analysis, or matrix factorization.

85. The method of claim 75, wherein said plurality of regulatory elements comprises a first set of regulatory elements having below-average enrichment efficiency and a second set of regulatory elements having above-average enrichment efficiency, and wherein said probe set comprises a first set of probe sequences that targets said first set of regulatory elements and a second set of probe sequences that targets said second set of regulatory elements.

86. The method of claim 75, further comprising quantifying sequencing reads of said plurality of regulatory elements to determine the availability of said plurality of regulatory elements.

87. The method of claim 75, further comprising determining a nucleosomal occupancy of said plurality of regulatory elements to determine the availability of said plurality of regulatory elements.

88. The method of claim 75, wherein said subject is a subject with cancer.

89. The method of claim 75, wherein said subject is a subject without cancer.

90. A system comprising a computer processor, wherein said computer processor is programmed to:
(a) enrich for nucleic acid sequences in a nucleic acid sample from a subject, wherein said nucleic acid sequences comprise at least a subset of a plurality of regulatory elements, thereby providing an enriched nucleic acid sample;
(b) sequence said enriched nucleic acid sample or a derivative thereof to generate a plurality of sequence reads comprising sequences that align with said subset of said plurality of regulatory elements;
(c) process said plurality of sequence reads to determine an expression profile of genes operably linked to said subset of said plurality of regulatory elements; and
(d) using at least said expression profile of genes to identify a disease in said subject at an accuracy of at least 90%.

91. The system of claim 90, wherein said regulatory elements are transcriptional start sites (TSS), enhancer sites, silencers, promoters, operators, untranslated regions (UTR), leader sequences (5′ UTR), trailer sequences (3′ UTR), terminators, or any combination thereof.

92. The system of claim 90, wherein said computer processor is further programmed to, prior to (b), process said nucleic acid sample with a plurality of barcodes.

93. The system of claim 92, wherein said plurality of barcodes comprises unique molecular identifiers.

94. The system of claim 90, wherein said regulatory elements are microRNA (miRNA) regulatory elements, messenger RNA (mRNA) regulatory elements, small interfering RNA (siRNA) regulatory elements, piwi-interacting RNA (piRNA) regulatory elements, small nucleolar RNA (snoRNA) regulatory elements, small nuclear RNA (snRNA) regulatory elements, extracellular RNA (exRNA) regulatory elements, small Cajal body-specific RNA (scaRNA) regulatory elements, non-coding RNA (ncRNA) regulatory elements, or any combination thereof.

95. The system of claim 90, wherein said processing of said plurality of sequence reads is against a reference sequence.

96. The system of claim 90, wherein said processing of said plurality of sequence reads is using statistics, mathematics, or biology.

97. The system of claim 90, wherein said processing of said plurality of sequence reads is a dimension reduction method.

98. The system of claim 97, wherein said dimension reduction method is principal component analysis, autoencoding, singular value decomposition, Fourier bases, wavelets, or discriminant analysis.

99. The system of claim 90, wherein said processing of said plurality of sequence reads comprises a supervised machine learning method, wherein said supervised machine learning method is a regression, support vector machine, tree-based method, neural network, or nearest neighbor method.

100. The system of claim 90, wherein said processing of said plurality of sequence reads comprises an unsupervised machine learning method, wherein said unsupervised machine learning method is clustering, neural network, principal component analysis, or matrix factorization.
